(19) United States
(12) Patent Application Publication
(10) Pub. No.: US 2007/0225528 A1
(43) Pub. Date: Sep. 27, 2007

(54) Process for Producing Optically Active Alcohol

(75) Inventors: Ryoji Noyori, Tokyo (JP); Takeshi Ohkuma, Hokkaido (JP); Kunihiko Tsutsumi, Tokyo (JP); Noriyuki Utsumi, Saitama (JP); Kunihiko Murata, Saitama (JP)

Correspondence Address: OLIFF & BERRIDGE, PLC, P.O. BOX 19928, ALEXANDRIA, VA, 22320, US

(73) Assignee: NAGOYA INDUSTRIAL SCIENCE RESEARCH CENTER, AICHI (JP)

(21) Appl. No.: 10/594,327

(22) PCT Filed: Mar. 29, 2004
(86) PCT No.: PCT/JP04/04422

§ 371 Date: Sep. 27, 2006

(51) Int. Cl. C07C 29/00

(52) U.S. Cl. 568/700

(57) ABSTRACT

A ruthenium complex RuCl[(S,S)-Tsdpen](p-cymene) represented by a formula below and a ketone compound are placed in a polar solvent, and the resulting mixture is mixed under pressurized hydrogen to hydrogenate the ketone compound and to thereby produce an optically active alcohol:

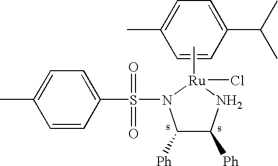

### TECHNICAL FIELD

The present invention relates to a process for producing an optically active alcohol in the presence of a ruthenium metal complex or the like as a catalyst.

### BACKGROUND ART

Various processes for producing optically active alcohols in the presence of metal complexes as catalysts have been reported to date. In particular, a process for synthesizing an optically active alcohol from a carbonyl compound in the presence of an asymmetric metal complex as a catalyst has been intently studied.

For example, Japanese Unexamined Patent Application Publication No. 2003-104993 reports several examples of producing optically active alcohols by hydrogenation of various ketone compounds in 2-propanol without addition of any base under pressurized hydrogen catalyzed by a tetrahydroborate of an asymmetric ruthenium metal complex that has a diamine compound and a diphosphine compound, such as BINAP (2,2′-bis(diphenylphosphino)-1,1′-binaphthyl) or the like, coordinated to ruthenium. In particular, corresponding optically active alcohols are produced from acetophenone, ethyl 4-acetylbenzoate, and 3-nonen-2-one.

Japanese Unexamined Patent Application Publication No. Hei11-322649 reports an example of producing a corresponding optically active alcohol by hydrogenation of m-trifluoromethylacetophenone in the presence of triethylamine and an azeotrope of formic acid and triethylamine while using as a catalyst an asymmetric ruthenium metal complex in which a diphenylethylenediamine having a sulfonyl group on the nitrogen and a benzene derivative are coordinated to ruthenium.

However, when the catalyst disclosed in Japanese Unexamined Patent Application Publication No. 2003-104993 is used, although an optically active alcohol can be produced from a ketone compound without any base, the yield or the enantiomeric excess is low for some reaction substrates. In Japanese Unexamined Patent Application Publication No. Hei11-322649, since triethylamine, which is an organic base, is necessary, production of an optically active alcohol from a reaction substrate, such as acetylene ketone which is unstable in the presence of bases, has been difficult.

### DISCLOSURE OF INVENTION

The present invention has been made to overcome the problems described above. An object of the present invention is to provide a process for producing an optically active alcohol from a ketone compound, hydrogenation of which has been difficult, in high yield and with high stereoselectivity.

To overcome these problems, the present inventors have studied the catalytic activity of many asymmetric ruthenium, rhodium, and iridium complexes, analyzed the principles of catalytic action, and developed a process of obtaining an optically active alcohol from a ketone compound, hydrogenation of which has been difficult, in high yield and with high stereoselectivity based on extensive studies.

The present invention provides a first process for producing an optically active alcohol, including placing a metal complex represented by formula (1) and a ketone compound in a polar solvent and stirring the mixture under pressurized hydrogen to hydrogenate the ketone compound and to thereby produce the optically active alcohol: 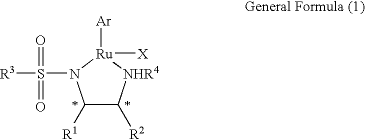
(where R¹ and R² may be the same or different and are each selected from the group consisting of an alkyl group, an optionally substituted phenyl group, an optionally substituted naphthyl group, and an optionally substituted cycloalkyl group, or together form an optionally substituted alicyclic ring;

R³ is one selected from the group consisting of an alkyl group, a perfluoroalkyl group, an optionally substituted naphthyl group, an optionally substituted phenyl group, and a camphor group;

R⁴ is a hydrogen atom or an alkyl group;

Ar is an optionally substituted benzene;

X is an anionic group; and

* represents an asymmetric carbon.)

According to this first process, since hydrogenation of a ketone compound proceeds under pressurized hydrogen, an optically active alcohol can be obtained from the ketone compound, hydrogenation of which has been difficult, in high yield and with high stereoselectivity.

The present invention also provides a second process for producing an optically active alcohol including placing a metal complex represented by formula (2) and a ketone compound in a polar solvent and stirring the mixture under pressurized hydrogen to hydrogenate the ketone compound and to thereby produce the optically active alcohol: 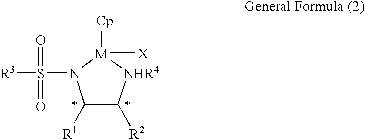
(where R¹ and R² may be the same or different and are each selected from the group consisting of an alkyl group, an optionally substituted phenyl group, an optionally substituted naphthyl group, and an optionally substituted cycloalkyl group, or together form an optionally substituted alicyclic ring;

R³ is one selected from the group consisting of an alkyl group, a perfluoroalkyl group, an optionally substituted naphthyl group, an optionally substituted phenyl group, and a camphor group;

R⁴ is a hydrogen atom or an alkyl group;

Cp is an optionally substituted cyclopentadiene;

M is rhodium or iridium;

X is an anionic group; and

* represents an asymmetric carbon.)

Also according to this second process, since hydrogenation of a ketone compound proceeds under pressurized hydrogen, an optically active alcohol can be obtained from the ketone compound, hydrogenation of which has been difficult, in high yield and with high stereoselectivity.

Examples of the alkyl group for R¹ and R² of general formula (1) or (2) include C₁-C₁₀ alkyl groups, such as a methyl group, an ethyl group, an n-propyl group, an isopropyl group, an n-butyl group, a sec-butyl group, and a tert-butyl group. Examples of the optionally substituted phenyl group include an unsubstituted phenyl group, an alkyl-containing phenyl group, such as a 4-methylphenyl group or a 3,5-dimethylphenyl group, a phenyl group having a halogen substituent, such as a 4-fluorophenyl group or a 4-chlorophenyl group, and an alkoxy-containing phenyl group, such as a 4-methoxyphenyl group. Examples of the optionally substituted naphthyl group include an unsubstituted naphthyl group, a 5,6,7,8-tetrahydro-1-naphthyl group, and a 5,6,7,8-tetrahydro-2-naphthyl group. Examples of the optionally substituted cycloalkyl group include a cyclopentyl group and a cyclohexyl group. Examples of the substituted or unsubstituted alicyclic ring formed by R¹ and R² include a cyclohexane ring formed by R¹ and R². R¹ and R² are preferably both a phenyl group or preferably form a cyclohexane ring by binding each other.

Examples of the alkyl group for R³ in general formula (1) or (2) include C₁-C₁₀ alkyl groups such as a methyl group, an ethyl group, an n-propyl group, an isopropyl group, an n-butyl group, a sec-butyl group, and a tert-butyl group. Examples of the perfluoroalkyl group include a trifluoromethyl group and a pentafluoroethyl group. Examples of the optionally substituted naphthyl group include an unsubstituted naphthyl group, a 5,6,7,8-tetrahydro-1-naphthyl group, and a 5,6,7,8-tetrahydro-2-naphthyl group. Examples of the optionally substituted phenyl group include an unsubstituted phenyl group, an alkyl-containing phenyl group, such as a 4-methylphenyl group, a 3,5-dimethylphenyl group, a 2,4,6-trimethylphenyl group, and a 2,4,6-triisopropylphenyl group, a phenyl group having a halogen substituent, such as a 4-fluorophenyl group or a 4-chlorophenyl group, and an alkoxy-containing phenyl group, such as a 4-methoxyphenyl group.

Examples of the alkyl group for R⁴ in general formula (1) or (2) include a methyl group and an ethyl group. R⁴ is preferably hydrogen.

Examples of Ar in general formula (1) include in addition to unsubstituted benzene, alkyl-containing benzene such as toluene, o-, m-, or p-xylene, o-, m-, or p-cymene, 1,2,3-, 1,2,4-, or 1,3,5-trimethylbenzene, 1,2,4,5- or 1,2,3,4-tetramethylbenzene, pentamethylbenzene, and hexamethylbenzene.

Examples of Cp in general formula (2) include, in addition to unsubstituted cyclopentadiene, alkyl-containing cyclopentadiene such as mono-, di-, tri-, tetra-, or pentamethylcyclopentadiene.

X in general formula (1) or (2) is an anionic group. Examples thereof include a fluorine group, a chlorine group, a bromine group, an iodine group, a tetrafluoroborate group, a tetrahydroborate group, a tetrakis[3,5-bis(trifluoromethyl)phenyl]borate group, an acetoxy group, a benzoyloxy group, a (2,6-dihydroxybenzoyl)oxy group, a (2,5-dihydroxybenzoyl)oxy group, a (3-aminobenzoyl)oxy group, a (2,6-methoxybenzoyl)oxy group, a (2,4,6-triisopropylbenzoyl)oxy group, a 1-naphthalenecarboxylic acid group, 2-naphthalenecarboxylic acid group, a trifluoroacetoxy group, a trifluoromethanesulfoxy group, and a trifluoromethanesulfonimide group. X is preferably a halogen group such as a fluorine group, a chlorine group, a bromine group, or an iodine group.

R¹, R², and R³ in general formula (1) or (2) may be the same or different and each preferably represent a phenyl group, a phenyl group containing a C₁-C₅ alkyl group, a phenyl group containing a C₁-C₅ alkoxy group, or a phenyl group containing a halogen substituent; and R⁴ is preferably a hydrogen atom. Since a bidentate ligand, an ethylene diamine derivative (R³SO₂NHCHR¹CHR²NHR⁴), is coordinated to ruthenium in general formula (1) and to rhodium or iridium in general formula (2), specific preferable examples of R¹ to R⁴ are described below as the examples of ethylenediamine derivatives. Namely, examples of the ethylenediamine derivatives include TsDPEN (N-(p-toluenesulfonyl)-1,2-diphenylethylenediamine, MsDPEN (N-methanesulfonyl-1,2-diphenylethylenediamine, N-methyl-N′-(p-toluenesulfonyl)-1,2-diphenylethylenediamine, N-(p-methoxyphenylsulfonyl)-1,2-diphenylethylenediamine, N-(p-chlorophenylsulfonyl)-1,2-diphenylethylenediamine, N-trifluoromethanesulfonyl-1,2-diphenylethylenediamine, N-(2,4,6-trimethylbenzenesulfonyl)-1,2-diphenylethylenediamine, N-(2,4,6-triisopropylbenzenesulfonyl)-1,2-diphenylethylene diamine, N-(4-tert-butylbenzenesulfonyl)-1,2-diphenylethylenediamine, N-(2-naphthylsulfonyl)-1,2-diphenylethylenediamine, N-(3,5-dimethylbenzenesulfonyl)-1,2-diphenylethylenediamine, N-pentamethylbenzenesulfonyl-1,2-diphenylethylenediamine, and 1,2-N-tosylcyclohexanediamine.

The present invention also provides a third process for producing an optically active alcohol including placing a metal complex represented by formula (3) and a ketone compound in a polar solvent and stirring the mixture under pressurized hydrogen to hydrogenate the ketone compound and to thereby produce the optically active alcohol: 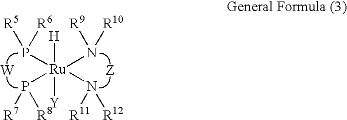
(where W is an optionally substituted bonding chain;

R⁵ to R⁸ may be the same or different and each represent an optionally substituted hydrocarbon group; R⁵ and R⁶ may bind each other to form an optionally substituted carbon chain ring; and R⁷ and R⁸ may bind each other to form an optionally substituted carbon chain ring;

R⁹ to R¹² may be the same or different and each represent a hydrogen atom or an optionally substituted hydrocarbon group;

Z is an optionally substituted hydrocarbon chain;

Y is an anionic group other than BH₄; and

each ligand of the ruthenium may be at any position.)

Also according to this third process, since hydrogenation of a ketone compound proceeds under pressurized hydrogen, an optically active alcohol can be obtained from the ketone compound, hydrogenation of which has been difficult, in high yield and with high stereoselectivity.

Examples of the optionally substituted hydrocarbon group for R⁵ to R⁸ of general formula (3) include saturated or unsaturated aliphatic or alicyclic hydrocarbons, monocyclic or polycyclic aromatic or aromatic aliphatic hydrocarbons, and these hydrocarbon groups with substituents. For example, selection may be made from hydrocarbon groups such as alkyl, alkenyl, cycloalkyl, cycloalkenyl, phenyl, naphthyl, and phenylalkyl, and hydrocarbon groups with various acceptable substituents such as alkyl, alkenyl, cycloalkyl, aryl, alkoxy, ester, acyloxy, a halogen atom, nitro, or cyano. When R⁵ and R⁶ or R⁷ and R⁸ bind each other to form an optionally substituted carbon chain ring, R⁵ and R⁶ or R⁷ and R⁸ may selected from those carbon chains having various acceptable substituents, such as alkyl, alkenyl, cycloalkyl, aryl, alkoxy, ester, acyloxy, a halogen atom, nitro, and cyano on the carbon chain.

W in general formula (3) is a bonding chain that may have a substituent. Examples of the bonding chain include divalent hydrocarbon chains (e.g., linear hydrocarbon chains such as —CH₂—, —(CH₂)₂—, —(CH₂)₃—, and —(CH₂)₄—; branched hydrocarbon chains such as —CH₂CH(CH₃)— and —CH(CH₃)CH(CH₃)—; and cyclic hydrocarbons such as —C₆H₄— and —C₆H₁₀—), divalent binaphthyl, divalent biphenyl, divalent paracyclophane, divalent bipyridine, and divalent heterocyclic rings. Of these, a binaphthyl group which is bonded to phosphorus atoms at 2-position and 2′-position and which may have a substituent in any other position is preferable. The bonding chain may include any one of various acceptable substituents such as alkyl, alkenyl, cycloalkyl, aryl, alkoxy, ester, acyloxy, a halogen atom, nitro, and cyano.

In general formula (3), a bidentate ligand, namely, a diphosphine derivative (R⁵R⁶P—W—PR⁷R⁸), is coordinated to ruthenium; therefore, specific preferable examples of R⁵ to R⁸ and W are described below as examples of diphosphine derivatives. That is, examples of the diphosphine derivatives include BINAP (2,2′-bis(diphenylphosphino) -1,1′-binaphthyl), TolBINAP (2,2′-bis[(4-methylphenyl)phosphino]-1,1′-binaphthyl), XylBINAP (2,2′-bis[(3,5-dimethylphenyl)phosphino]-1,1′-binaphthyl), 2,2′-bis[(4-tert-butylphenyl)phosphino]-1,1′-binaphthyl), 2,2′-bis[(4-isopropylphenyl)phosphino]-1,1′-binaphthyl), 2,2′-bis[(naphthalen-1-yl)phosphino]-1,1′-binaphthyl), 2,2′-bis[(naphthalen-2-yl)phosphino]-1,1′-binaphthyl), BICHEMP (2,2′-bis(dicyclohexylphosphino)-6,6′-dimethyl-1,1′-biphenyl), BPPFA (1-[1,2-bis-(diphenylphosphino)ferrocenyl]ethylamine), CHIRAPHOS (2,3-bis(diphenylphosphino)butane, CYCPHOS (1-cyclohexyl-1,2-bis(diphenylphosphino)ethane), DEGPHOS (1-substituted-3,4-bis(diphenylphosphino)pyrrolidine), DIOP (2,3-isopropylidene-2,3-dihydroxy-1,4-bis((diphenylphosphino)butane), SKEWPHOS (2,4-bis(diphenylphosphino)pentane), DuPHOS (substituted-1,2-bis(phosphorano)benzene), DIPAMP (1,2-bis[(o-methoxyphenyl)phenylphosphino]ethane), NORPHOS (5,6-bis(diphenylphosphino)-2-norbornene), PROPHOS (1,2-bis(diphenylphosphino)propane, PHANEPHOS (4,12-bis(diphenylphosphino)-[2,2′]-paracyclophane), and substituted-2,2′-bis(diphenylphosphino)-1,1′-bipyridine).

Examples of the hydrocarbon group for R⁹ to R¹² in general formula (3) include C₁ to C₁₀ hydrocarbon groups such as a methyl group, an ethyl group, a propyl group, and a benzyl group. These hydrocarbon groups may have various acceptable substituents such as alkyl, alkenyl, cycloalkyl, aryl, alkoxy, ester, acyloxy, a halogen atom, nitro, and cyano.

Examples of the hydrocarbon chain for Z in general formula (3) include linear hydrocarbon chains such as —CH₂—, —(CH₂)₂—, —(CH₂)₃—, and —(CH₂)₄—; branched hydrocarbon chains such as —CH₂CH(CH₃)— and —CH(CH₃)CH(CH₃)—; and cyclic hydrocarbons such as —C₆H₄— and —C₆H₁₀—. These hydrocarbon chains may include various acceptable substituents such as alkyl, alkenyl, cycloalkyl, aryl, alkoxy, ester, acyloxy, a halogen atom, nitro, and cyano. Of these, a phenyl group is preferable as the substituent.

In general formula (3), a bidentate ligand, namely, a diamine derivative (R⁹R¹⁰N-Z-NR¹¹R¹²), is coordinated to ruthenium; therefore, specific preferable examples of R⁹ to R¹² and Z are described below as examples of diamine derivatives. That is, examples of the diamine derivative include DPEN (1,2-diphenylethylenediamine), N-methyl-1,2-diphenylethylenediamine, N,N′-dimethyl-1,2-diphenylethylenediamine, 1,2-cyclohexanediamine, DAIPEN (1-isopropyl-2,2-di(p-methoxyphenyl)ethylenediamine), 1,2-cycloheptanediamine, 2,3-dimethylbutanediamine, 1-methyl-2,2-diphenylethylenediamine, 1-isopropyl-2,2-diphenylethylenediamine, 1-methyl-2,2-di(p-methoxyphenyl)ethylenediamine, 1-ethyl-2,2-di(p-methoxyphenyl)ethylenediamine, 1-phenyl-2,2-di(p-methoxyphenyl)ethylenediamine, 1-benzyl-2,2-di(p-methoxyphenyl)ethylenediamine, and 1-isobutyl-2,2-di(p-methoxyphenyl)ethylenediamine. Of these, DPEN or DAIPEN is preferable. Among these, an optically active diamine derivative is preferable. The optically active diamine derivative is not limited to those described above, and various optically active propanediamine, butanediamine, phenylenediamine, and cyclohexanediamine derivatives may be used.

In general formula (3), Y represents an anionic group other than a tetrahydroborate group (BH₄), and examples thereof include a fluorine group, a chlorine group, a bromine group, an iodine group, an acetoxy group, a benzoyloxy group, a (2,6-dihydroxybenzoyl)oxy group, a (2,5-dihydroxybenzoyl)oxy group, a (3-aminobenzoyl)oxy group, a (2,6-methoxybenzoyl)oxy group, a (2,4,6-triisopropylbenzoyl)oxy group, a l-naphthalenecarboxylic acid group, a 2-naphthalenecarboxylic acid group, a trifluoroacetoxy group, a trifluoromethanesulfoxy group, a trifluoromethanesulfonimide group, and a tetrafluoroborate group (BF₄). Of these, a halogen group such as a fluorine group, a chlorine group, a bromine group, or an iodine group is preferable as Y.

The metal complexes represented by general formulae (1) to (3) may each include one or more coordinating organic solvents. Examples of the coordinating organic solvent include aromatic hydrocarbon solvents such as toluene and xylene, aliphatic hydrocarbon solvents such as pentane and hexane, halogen-containing hydrocarbon solvents such as methylene chloride, ether solvents such as ether and tetrahydrofuran, alcohol solvents such as methanol, ethanol, 2-propanol, butanol, and benzyl alcohol, ketone solvents such as acetone, methyl ethyl ketone, and cyclohexyl ketone, and heteroatom-containing organic solvents such as acetonitrile, dimethylformamide (DMF), N-methylpyrrolidone, dimethyl sulfoxide (DMSO), and triethylamine.

Methods for preparing ruthenium, rhodium, and iridium complexes represented by general formulae (1) and (2) are disclosed in Angew. Chem., Int. Ed. Engl. Vol. 36, p. 285 (1997), J. Org. Chem. Vol. 64, p. 2186 (1999), and the like. That is, synthesis is possible through reaction between a sulfonyl diamine ligand and a ruthenium, rhodium, or iridium complex having a ligand X. Alternatively, synthesis is possible through reaction between HX and a metal amide complex having a sulfonyl diamine ligand.

The method of preparing a ruthenium complex represented by general formula (3) is disclosed in Angew. Chem., Int. Ed. Engl. Vol. 37, p. 1703 (1998), Organometallics vol. 21, p. 1047 (2001), or the like. That is, a ruthenium hydride complex having a ligand X is reacted with a diphosphine ligand and then with a diamine ligand. Alternatively, a ruthenium halide is reacted with a diphosphine ligand and then with a diamine ligand to prepare a ruthenium halide complex having a diphosphine ligand and a diamine ligand, and then the ruthenium halide complex is reduced to prepare the target ruthenium complex.

Examples of the ruthenium complex which is used as the starting material for the ruthenium complex represented by general formula (1) include inorganic ruthenium compounds such as ruthenium(III) chloride hydrate, ruthenium(III) bromide hydrate, and ruthenium(III) iodide hydrate; diene-liganded ruthenium compounds, such as a [ruthenium dichloride(norbornadiene)]polynuclear complex, a [ruthenium dichloride(cycloocta-1,5-diene)]polynuclear complex, and bis(methylallyl)ruthenium(cycloocta-1,5-diene); aromatic-compound-liganded ruthenium complexes such as a [ruthenium dichloride(benzene)]polynuclear complex, a [ruthenium dichloride(p-cymene)]polynuclear complex, a [ruthenium dichloride(trimethylbenzene)]polynuclear complex, and a [ruthenium dichloride(hexamethylbenzene)]polynuclear complex; phosphine-liganded complexes such as dichlorotris(triphenylphosphine)ruthenium; ruthenium dichloride(dimethylformamide)₄; and chlorohydridetris(triphenylphosphine)ruthenium. The ruthenium complex may be any other ruthenium complex that has a ligand substitutable with an optically active diphosphine compound and an optically active diamine compound and is not limited to those described above. For example, various ruthenium complexes disclosed in COMPREHENSIVE ORGANOMETALLIC CHEMISTRY II Vol. 7, pp. 294-296 (PERGAMON) can be used as the starting material.

Examples of the rhodium and iridium complexes that can be used as the starting materials for the asymmetric rhodium complex and the asymmetric iridium complex represented by general formula (2) include inorganic ruthenium compounds such as rhodium( III) chloride hydrate, rhodium( III) bromide hydrate, and rhodium(III) iodide hydrate, a [pentamethylcyclopentadienylrhodium dichloride]polynuclear complex, a [pentamethylcyclopentadienylrhodium dibromide]polynuclear complex, and a [pentamethylcyclopentadienylrhodium diiodide]polynuclear complex.

The reaction between the starting materials, i.e., the ruthenium, rhodium, and iridium complexes, and the ligands are carried out in at least one solvent selected from the group consisting of aromatic hydrocarbon solvents such as toluene and xylene, aliphatic hydrocarbon solvents such as pentane and hexane, halogen-containing hydrocarbon solvents such as methylene chloride, ether solvents such as ether and tetrahydrofuran, alcohol solvents such as methanol, ethanol, 2-propanol, butanol, and benzyl alcohol, and heteroatom-containing organic solvents such as acetonitrile, DMF, N-methylpyrrolidone, and DMSO, at a reaction temperature of 0° C. to 200° C. to thereby yield a metal complex.

According to each of the first to third processes of the present invention, a metal complex represented by general formula (1) to (3) and ketone are placed in a polar solvent and mixed under pressurized hydrogen to hydrogenate the ketone compounds. The pressure of the hydrogen is preferably 1 to 200 atm and more preferably 5 to 150 atm from the standpoint of economy. The reaction can be carried out in the range of −50° C. to 100° C., preferably in the range of −30° C. to 50° C., and most preferably in the range of 20° C. to 50° C. The reaction time differs depending on reaction conditions including the reaction substrate concentration, temperature, pressure, and the like. Typically, the reaction is completed in several minutes to several days and frequently in 5 to 24 hours. The purification of the reaction product can be conducted by a known method such as column chromatography, distillation, recrystallization or the like. In using the metal complex represented by general formula (1) or (2), an amide complex corresponding to the metal complex represented by general formula (1) or (2) may be added (e.g., metal complex:amide complex=1.0:0 to 1.0 molar equivalent), or, HX (X as defined above) may be added to the metal complex represented by general formula (1) or (2) (e.g., metal complex:HX=1.0:0 to 0.5 molar equivalents). Furthermore, hydrogenation reaction of the ketone compound may be carried out in the reaction system after preparation of the metal catalyst represented by general formula (1) or (2) from the corresponding amide complex and HX (e.g., amide complex:HX=1.0:0.5 to 1.5 molar equivalents).

Examples of the polar solvent used in the first to third processes of the present invention include alcohol solvents such as methanol, ethanol, 2-propanol, 2-methyl-2-propanol, and 2-methyl-2-butanol, ether solvents such as tetrahydrofuran (THF) and diethyl ether, and heteroatom-containing solvents such as DMSO, DMF, and acetonitrile. These solvents may be used alone or in combination. A mixed solvent containing the polar solvent above and a solvent other than those described above may also be used. Among these polar solvents, alcohol solvents are preferable, methanol and ethanol are more preferable, and methanol is most preferable.

The amounts of the metal complexes represented by general formulae (1) to (3) used in the first to third processes of the present invention are preferably in the range of S/C of 10 to 100,000 and more preferably in the range of S/C of 50 to 10,000, where S/C is a molar ratio of the ketone compound to the metal complex, S representing the substrate and C representing the catalyst.

In the reaction systems according to the first to third processes of the present invention, a salt of an organic or inorganic substance may be added if necessary. Examples of the salt include ionic salts such as lithium perchlorate, sodium perchlorate, magnesium perchlorate, barium perchlorate, calcium perchlorate, lithium hexafluorophosphate, sodium hexafluorophosphate, magnesium hexafluorophosphate, calcium hexafluorophosphate, lithium tetrafluoroborate, sodium tetrafluoroborate, magnesium tetrafluoroborate, calcium tetrafluoroborate, lithium tetraphenylborate, sodium tetraphenylborate, magnesium tetraphenylborate, and calcium tetraphenylborate. To the metal complex, 1 to 1,000 molar equivalents of the salt may be added to hydrogenate the ketone. Preferably, 10 to 200 molar equivalents of a perchlorate is used relative to the metal complex.

The asymmetric carbons in the metal complexes represented by general formulae (1) to (3) of the first to third processes of the present invention can be obtained as either the (R) isomer or the (S) isomer. By selecting either one of the (R) and (S) isomers, the target (R) or (S) isomer of an optically active alcohol can be obtained at high selectivity.

According to the first to third processes of the present invention, it is not essential to add a base to the reaction system. Hydrogenation reaction of the ketone compound rapidly proceeds without addition of the base. However, this should not be taken to exclude addition of the base; for example, a small amount of a base may be added depending on the reaction substrate.

As described above, the processes for producing an optically active alcohol according to the first to third processes of the present invention do not essentially require a base to conduct hydrogenation of the ketone compound. Thus, a ketone compound that is unstable in the presence of bases can be hydrogenated to obtain a corresponding optically active alcohol. In particular, a cyclic ketone can be hydrogenated to give an optically active cyclic alcohol; a ketone having an olefin moiety or an acetylene moiety (especially a ketone in which the α,β-bond is the olefin moiety or acetylene moiety) can be hydrogenated to give an optically active alcohol having an olefin moiety or an acetylene moiety; a ketone having a hydroxyl group can be hydrogenated to give an optically active alcohol having a hydroxyl group; a ketone having a halogen substituent (especially a ketone having a halogen substituent at the a-position) can be hydrogenated to give an optically active alcohol having a halogen substituent; a chromanone derivative can be hydrogenated to give an optically active chromanol; a diketone can be hydrogenated to give an optically active diol; a ketoester can be hydrogenated to give an optically active hydroxy ester; and a ketoamide can be hydrogenated to give an optically active hydroxyamide. The representative examples of the ketone compounds applicable to the first and third processes of the present invention are shown in FIGS. 1 to 7.

### BRIEF DESCRIPTION OF THE DRAWINGS

FIG. 1 is a first illustration that shows the structures of ketone compounds to which the process of producing an optically active alcohol of the present invention is applicable;

FIG. 2 is a second illustration that also shows structures of ketone compounds;

FIG. 3 is a third illustration that also shows structures of ketone compounds;

FIG. 4 is a fourth illustration that also shows structures of ketone compounds;

FIG. 5 is a fifth illustration that also shows structures of ketone compounds;

FIG. 6 is a sixth illustration that also shows structures of ketone compounds;

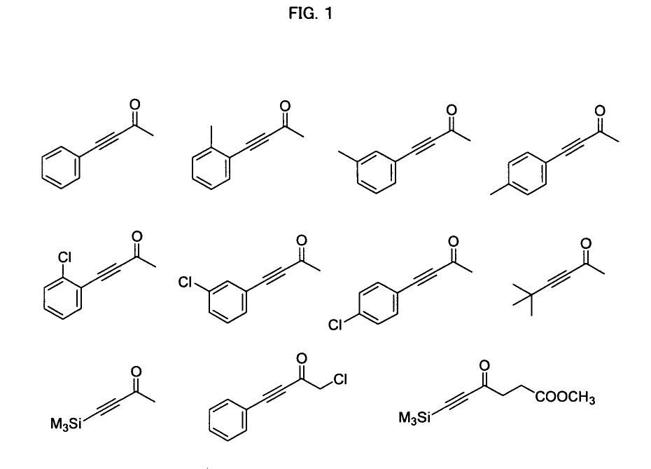

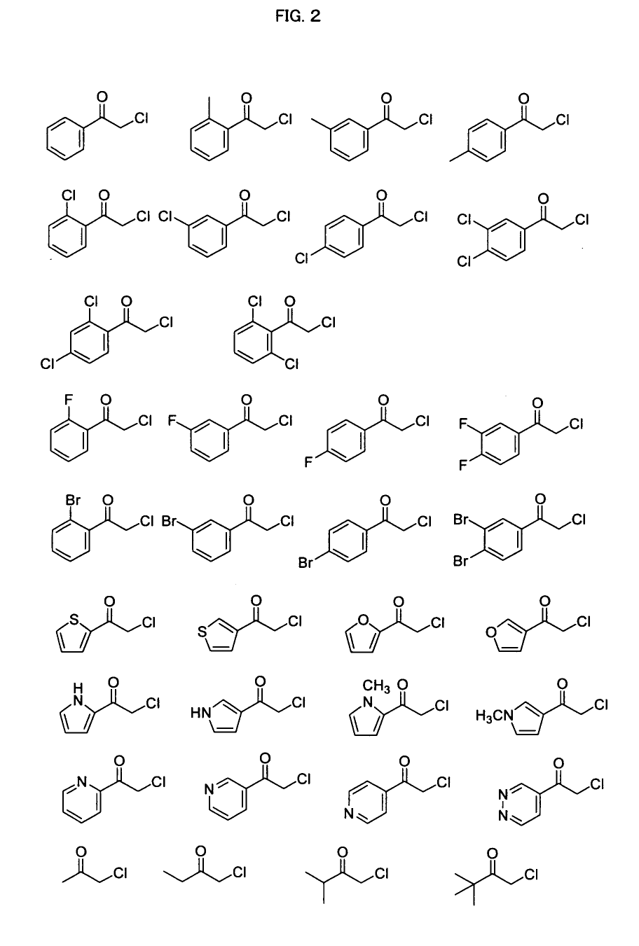

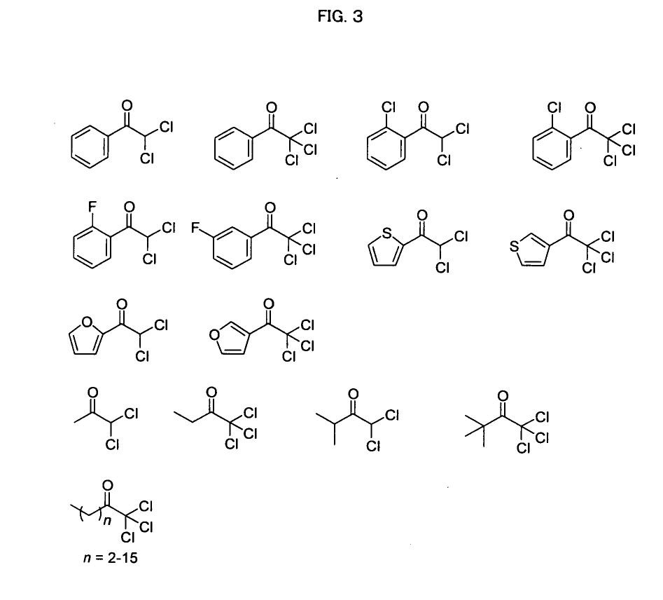

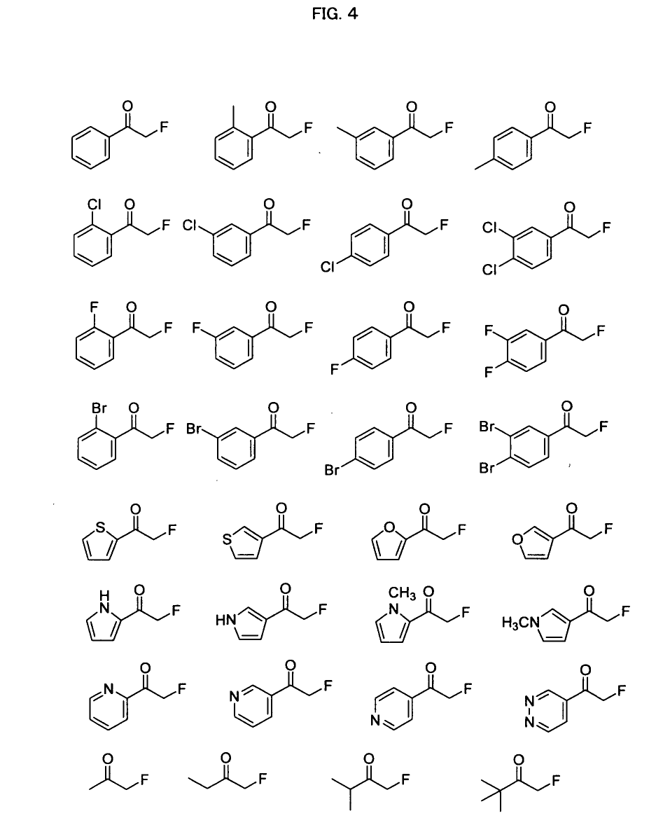

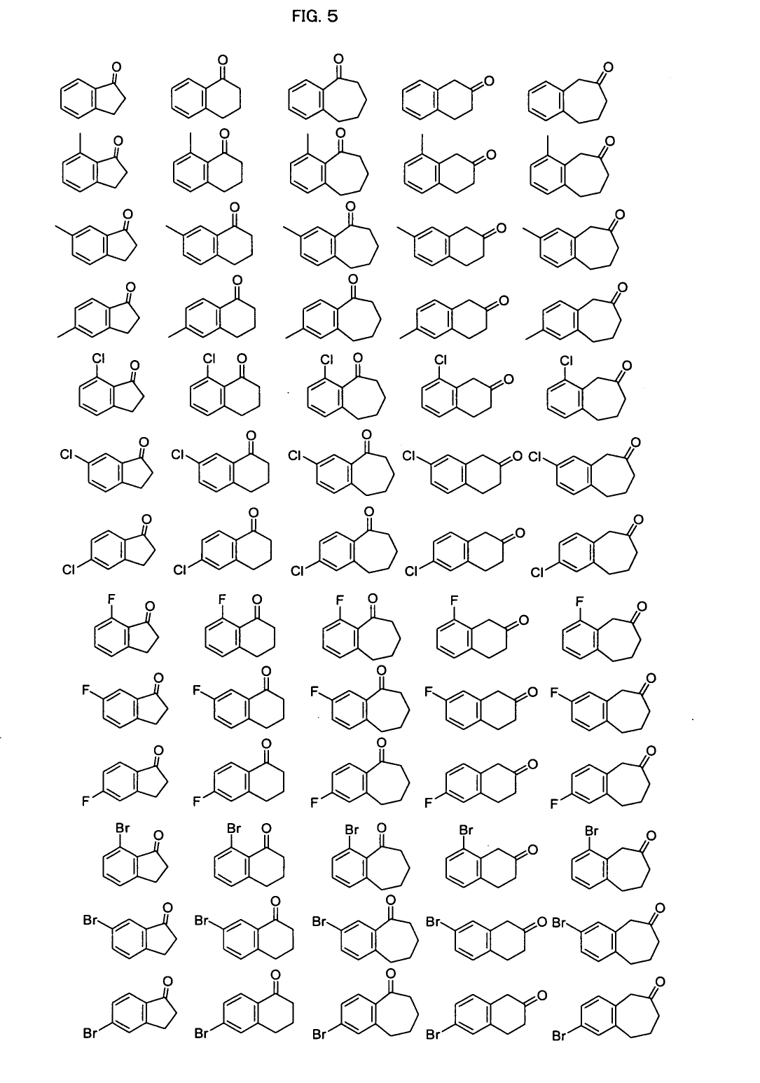

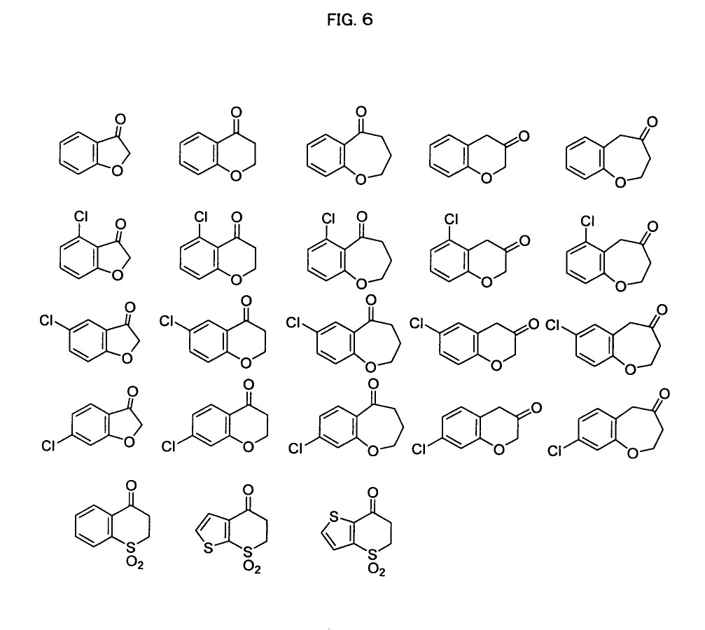

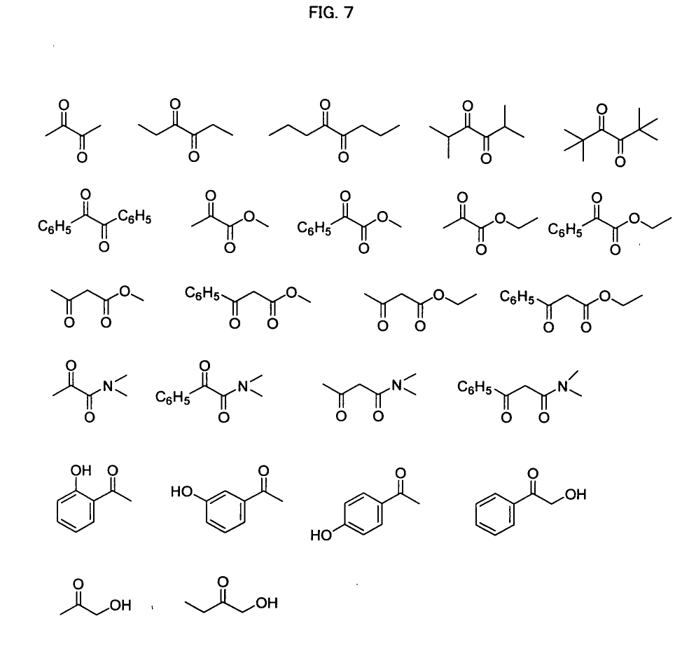

and FIG. 7 is a seventh illustration that also shows structures of ketone compounds.

### EXAMPLES

Hydrogenation of a carbonyl compound of the present invention may be conducted in a batch or continuous-flow system. Examples are described below to further describe the present invention in detail. It is to be understood that the present invention is not limited by the examples described below.

In the examples described below, the solvent used for the reaction was dried and degassed. For NMR measurements, JNM-LA400 (400 MHz, produced by JEOL Ltd.) and JNM-LA500 (500 MHz, produced by JEOL Ltd.) were used. For ¹H-NMR, tetramethylsilane (TMS) was used as the internal standard, and for ³¹P-NMR, 85% phosphoric acid was used as the external standard. For these signals, δ=0 (δ denotes the chemical shift) was assumed. Optical purity was measured by gas chromatography (GC) or high-performance liquid chromatography (HPLC). GC measurement was conducted using Chirasil-DEX CB (0.25 mm×25 m, DF=0.25 am) (produced by CHROMPACK), and HPLC measurement was conducted using a chiral compound isolation column (produced by Daicel). The metal complex represented by general formula (1) was synthesized by the technique disclosed in Angew. Chem., Int. Ed. Engl. Vol. 36, p. 285 (1997), the metal complex represented by general formula (2) was synthesized by the technique disclosed in J. Org. Chem. Vol. 64, p. 2186 (1999), and the metal complex represented by general formula (3) was synthesized by the technique disclosed in Angew. Chem., Int. Ed. Engl. Vol. 37, p. 1703 (1998) and Organometallics Vol. 21, p.1047 (2001).

### Example 1

An example of synthesizing (S)-4-phenyl-3-butyn-2-ol by hydrogenation of 4-phenyl-3-butyn-2-one is described below. A 50 mL stainless steel autoclave was charged with a ruthenium complex, RuCl[(S,S)-Tsdpen](p-cymene), (1.6 mg, 0.0025 mmol), followed by argon substitution. 4-Phenyl-3-butyn-2-one (0.291 mL, 2 mmol) and methanol (5 mL) were added. After pressurization with hydrogen, substitution was conducted five times. Hydrogen was charged to 50 atm to initiate reaction. After the reaction mixture was stirred for 11 hours at 30° C, the reaction pressure was reduced to normal. The product was analyzed by ¹H-NMR and HPLC reporting synthesis of (S)-4-phenyl-3-butyn-2-ol in 90% ee and 63% yield. For the purpose of this description, in the nomenclature of the ruthenium complex, the metal atom, the anionic group, the diamine ligand, and the arene ligand are presented in this order from the left (see formula (4) below):

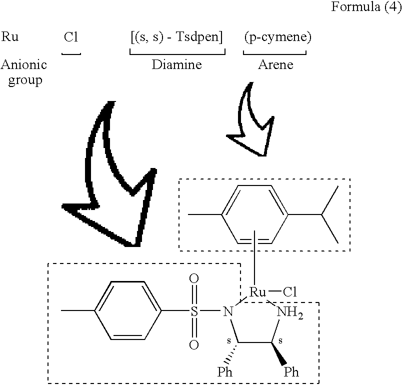

### Comparative Example 1

Reaction was conducted under the same conditions as in EXAMPLE 1 but without pressurization with hydrogen. The target substance was not obtained.

### Examples 2-10

Reaction was conducted under the same conditions as in EXAMPLE 1 but with different catalysts and/or hydrogen pressures to synthesize (S)-4-phenyl-3-butyn-2-ol. The results are shown in Table 1.

TABLE 1

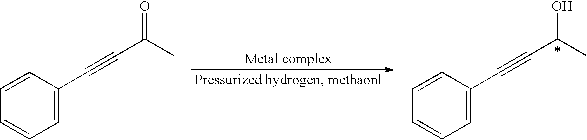

| Examples | chiral Ru cat | H₂ (atm) | yield (%) | ee (%) | config |
|---|---|---|---|---|---|
| 2 | RuCl[(S,S)-Tsdpen](p-cymene) | 9 | 18 | 81 | S |
| 3 | RuCl[(S,S)-Tsdpen](dmb) | 50 | 32 | 91 | S |
| 4 | RuCl[(S,S)-Tsdpen](mesitylene) | 50 | 100 | 79 | S |
| 5 | RuCl[(S,S)-Tsdpen](teb) | 50 | 61 | 91 | S |
| 6 | RuCl[(S,S)-Tsdpen](durene) | 50 | 29 | 71 | S |
| 7 | RuCl[(S,S)-Tsdpen](pmb) | 50 | 30 | 89 | S |
| 8 | RuCl[(S,S)-Tsdpen](hmb) | 50 | 78 | 88 | S |
| 9 | RuCl[(S,S)-Msdpen](p-cymene) | 50 | 78 | 88 | S |
| 10 | RuCl[(S,S)-(5,6,7,8-tetrahydronaphthalene- | 50 | 69 | 91 | S |
| | 2-yl)sulfonyl-dpen](p-cymene) | | | | |

Conditions: chiral Ru cat 0.0025 mmol,
CH₃OH 5 ml, S/C = 800, temp 30° C., time 11 h,
[ketone] = 0.4 M,
dmb: 1,4-dimethylbenzene,
teb: 1,3,5-triethylbenzene,
durene: 1,2,4,5-tetramethylbenzene,
pmb: pentamethylbenzene,
hmb: hexamethylbenzene.

### Examples 11-19

Reaction was conducted under the same conditions as in EXAMPLE 1 but with different substrate concentrations, reaction temperature, and/or additives to synthesize (S)-4-phenyl-3-butyn-2-ol. The results are shown in Table 2.

TABLE 2

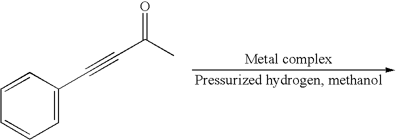

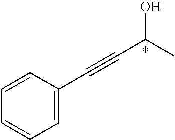

| Examples | additive | temp, ° C. | yield (%) | ee (%) | config |
|---|---|---|---|---|---|
| 11 | — | 50 | 50 | 87 | S |
| 12^{a} | — | 30 | 27 | 75 | S |
| 13^{b} | — | 30 | 33 | 88 | S |
| 14 | NaClO₄ | 30 | 88 | 92 | S |
| | 0.125 mmol | | | | |
| 15 | LiClO₄ | 30 | 80 | 92 | S |
| | 0.125 mmol | | | | |
| 16 | KClO₄ | 30 | 64 | 92 | S |
| | 0.125 mmol | | | | |
| 17 | BaClO₄ | 30 | 69 | 93 | S |
| | 0.125 mmol | | | | |
| 18 | NaPF₆ | 30 | 75 | 90 | S |
| | 0.125 mmol | | | | |
| 19 | NaBF₄ | 30 | 77 | 93 | S |
| | 0.125 mmol | | | | |

Conditions:
[ketone] = 0.4 M in CH₃OH, RuCl[(S,S)-Tsdpen](p-cymene) 0.0025 mmol, S/C = 800, H₂ 50 atm, time 11 h, solvent 5 ml,
^{a}[ketone] = 0.1 M,
^{b}[ketone] = 1.0 M.

### Examples 20-26

Reaction was conducted under the same conditions as in EXAMPLE 1 but with different catalysts and solvents and use of additives to synthesize (S)-4-phenyl-3-butyn-2-ol. The results are shown in Table 3.

TABLE 3

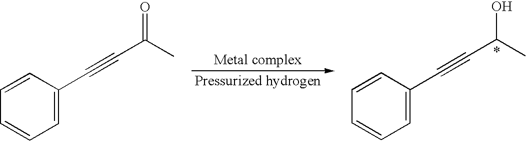

| Examples | chiral Ru cat | solvent | additive | yield (%) | ee (%) | config |
|---|---|---|---|---|---|---|
| 20 | RuCl[(S,S)-Tsdpen](p-cymene) | CH₃OH:H₂O = | NaClO₄ | 79 | 89 | S |
| | | 99:1 | 0.125 mmol | | | |
| 21 | RuCl[(S,S)-Tsdpen](p-cymene) | CH₃OH:THF = | NaClO₄ | 53 | 93 | S |
| | | 80:20 | 0.125 mmol | | | |
| 22 | RuCl[(S,S)-Tsdpen](p-cymene) | DMF:H₂O = | NaClO₄ | 37 | 92 | S |
| | | 80:20 | 0.125 mmol | | | |
| 23 | RuCl[(S,S)-Tsdpen](p-cymene) | CH₃OH | NaClO₄ | 68 | 92 | S |
| | | | 0.025 mmol | | | |
| 24 | RuCl[(S,S)-Tsdpen](p-cymene) | CH₃OH | NaClO₄ | 64 | 92 | S |
| | | | 2.5 mmol | | | |
| 25 | RuCl[(S,S)-Tsdpen](mesitylene)^{a} | CH₃OH | NaClO₄ | 69 | 90 | S |
| | | | 0.125 mmol | | | |
| 26 | RuCl[(S,S)-Tsdpen](mesitylene)^{b} | CH₃OH | NaClO₄ | 90 | 94 | S |
| | | | 0.125 mmol | | | |

Conditions:
[ketone] = 0.4 M, chiral Ru cat 0.0025 mmol, S/C = 800, H₂ 50 atm, temp 30° C., time 11 h, solvent 5 ml,
^{a}S/C = 2000.
^{b}S/C = 2000, H₂ 100 atm.

### Example 27

An example of synthesizing (S)-indanol by hydrogenation of 1-indanone is described below. A 50 mL stainless steel autoclave was charged with a ruthenium complex, RuCl[(S,S)-Tsdpen](p-cymene), (1.6 mg, 0.0025 mmol) and 1-indanone (330 mg, 2.5 mmol), followed by argon substitution. Methanol (5 mL) was added. Hydrogen was pressurized and substitution was conducted five times. Hydrogen was charged to 50 atm to initiate reaction. After the reaction mixture was stirred for 11 hours at 30° C., the reaction pressure was reduced to normal. The product was analyzed by ¹H-NMR and HPLC reporting synthesis of (S)-indanol in 98% ee and 48% yield.

### Examples 28-31

Reaction was conducted under the same conditions as in EXAMPLE 27 but with different catalysts, solvents, hydrogen pressures, and reaction times, and use of additives to synthesize an optically active indanol. The results are shown in Table 4.

TABLE 4

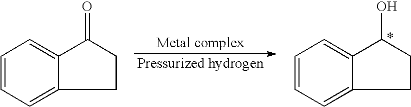

| Examples | chiral Ru cat | solvent | H₂ (atm) | yield (%) | ee (%) | config |
|---|---|---|---|---|---|---|
| 28 | RuCl[(S,S)-Tsdpen](mesitylene) | CH₃OH | 50 | 89 | 98 | S |
| 29 | RuCl[(S,S)-Tsdpen](mesitylene) | C₂H₅OH | 50 | 20 | 76 | S |
| 30 | RuCl[(S,S)-Tsdpen](mesitylene) | CH₃OH | 100 | 86 | 98 | S |
| 31 | RuCl[(S,S)-Tsdpen](mesitylene)^{a} | CH₃OH | 50 | 98 | 98 | S |

Conditions:
chiral Ru cat 0.0025 mmol, solvent 5 ml, S/C = 1000, temp 30° C., time 11 h,
[ketone] = 0.5 M.
^{a}24 h.

### Example 32

An example of synthesizing optically active 2-chloro-1-phenylethanol by hydrogenation of α-chloroacetophenone is described below. A 50 mL stainless steel autoclave was charged with a ruthenium complex, RuCl[(S,S)-Tsdpen](mesitylene) (1 mg, 0.0016 mmol) and α-chloroacetophenone (247 mg, 1.6 mmol). After argon substitution, methanol (3.2 mL) was added. Hydrogen was pressurized and substitution was conducted five times. Hydrogen was charged to 50 atm to initiate reaction. After stirring for 24 hours at 30° C., the reaction pressure was reduced to normal. The product was analyzed by ¹H-NMR and GC reporting synthesis of (R)-2-chloro-1-phenylethanol in 98% ee and 100% yield.

### Example 33

An example of synthesizing optically active 2-chloro-1-phenylethanol by hydrogenation of α-chloroacetophenone is described below. A 50 mL stainless steel autoclave was charged with a ruthenium complex, RuCl[(S,S)-Tsdpen](mesitylene) (1 mg, 0.0016 mmol), and a-chloroacetophenone (1235 mg, 8.0 mmol). After argon substitution, methanol (16.0 mL) was added. Hydrogen was pressurized and substitution was conducted five times. Hydrogen was charged to 100 atm to initiate reaction. After stirring for 22 hours at 30° C., the reaction pressure was reduced to normal. The product was analyzed by ¹H-NMR and GC reporting synthesis of (R)-2-chloro-1-phenylethanol in 97% ee and 85% yield.

### Example 34-40

Reaction was conducted under the same conditions as in EXAMPLE 32 but with different catalysts, hydrogen pressures, and reaction times to synthesize (R)-2-chloro-1 phenylethanol. The results are shown in Table 5.

TABLE 5

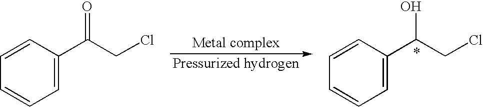

| Examples | chiral Ru cat | S/C | H₂ (atm) | yield (%) | ee (%) | config |
|---|---|---|---|---|---|---|
| 34 | RuCl[(S,S)-Tsdpen](p-cymene) | 1000 | 50 | 48 | 92 | R |
| 35 | RuCl[(S,S)-Tsdpen](mesitylene) | 1500 | 50 | 100 | 98 | R |
| 36 | RuCl[(S,S)-Tsdpen](mesitylene) | 2000 | 50 | 88 | 98 | R |
| 37 | RuCl[(S,S)-Tsdpen](mesitylene) | 2000 | 100 | 100 | 98 | R |
| 38 | RuCl[(S,S)-Tsdpen](mesitylene) | 3000 | 100 | 100 | 97 | R |
| 39 | RuCl[(S,S)-Tsdpen](mesitylene) | 4000 | 100 | 96 | 98 | R |
| 40 | RuCl[(S,S)-Tsdpen](mesitylene)^{a} | 5000 | 50 | 46 | 97 | R |

Conditions:
chiral Ru cat 0.0016 mmol, solvent CH₃OH, temp 30° C., time 24 h,
[ketone] = 0.5 M.
^{a}15 h.

### Example 41

An example of synthesizing optically active 2-chloro-1-phenylethanol by hydrogenation of α-chloroacetophenone is described below. Reaction was conducted under the same conditions as those of EXAMPLE 32 except that the reaction was conducted in the presence of a catalyst prepared from a ruthenium complex, Ru[(S,S)-Tsdpen](p-cymene), and HBF₄ and in a methanol/tert-butyl alcohol (1:1) mixture under a hydrogen pressure of 50 atm. As a result, (R)-2-chloro-1-phenylethanol was obtained in 95% ee and 100% yield.

### Example 42

An example of synthesizing optically active 2-chloro-1-phenylethanol by hydrogenation of α-chloroacetophenone is described below. Reaction was conducted under the same conditions as those of EXAMPLE 32 except that ruthenium complex CpRhCl[(S,S)-Tsdpen] (Cp: pentamethylcyclopentadiene) was used as a catalyst and the reaction was conducted for 11 hours. As a result, (R)-2-chloro-1-phenylethanol was obtained in 93% ee and 44% yield. Note that in the nomenclature of this ruthenium complex, the cyclopentadiene ligand, the metal atom, the anionic group, and the diamine ligand are presented in this order from the left (see formula (5) below):

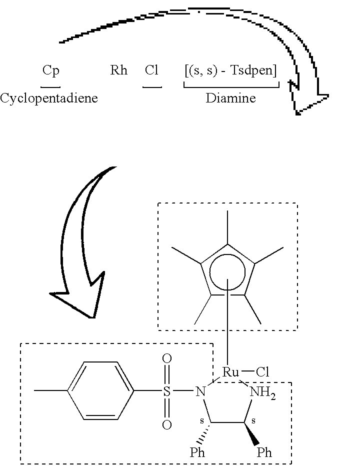

### Example 43

An example of synthesizing optically active 2-chloro-1-(p-methoxyphenyl)ethanol by hydrogenation of α-chloro-p-methoxyacetophenone is described below. A 50 mL stainless-steel autoclave was charged with a ruthenium complex, RuCl[(S,S)-Tsdpen](mesitylene) (1 mg, 0.0016 mmol), α-chloro-p-methoxyacetophenone (1477 mg, 8.0 mmol), and NaClO₄ (10 mg, 0.08 mmol). After argon substitution, methanol (16.0 mL) was added. Hydrogen was pressurized, and substitution was conducted five times. Hydrogen was charged to 100 atm to initiate reaction. After stirring for 24 hours at 30° C., the reaction pressure was reduced to normal. The product was analyzed by ¹H-NMR and GC reporting synthesis of (R)-2-chloro-1-(p-methoxyphenyl)ethanol in 98% ee and 93% yield.

### Example 44

An example of synthesizing optically active 2-chloro-1-(p-methoxyphenyl)ethanol by hydrogenation of α-chloro-p-methoxyacetophenone is described below. A 50 mL stainless steel autoclave was charged with a ruthenium complex, RuCl[(S,S)-Tsdpen](mesitylene) (1 mg, 0.0016 mmol), α-chloro-p-chloroacetophenone (605 mg, 3.2 mmol), and NaClO₄ (10 mg, 0.08 mmol). After argon substitution, methanol (6.4 mL) was added. Hydrogen was pressurized, and substitution was conducted five times. Hydrogen was charged to 100 atm to initiate reaction. After stirring for 24 hours at 30° C., the reaction pressure was reduced to normal. The product was analyzed by ¹H-NMR and GC reporting synthesis of (R)-2-chloro-1-(p-chlorophenyl)ethanol in 95% ee and 93% yield.

### Example 45

An example of synthesizing optically active 4-chromanol by hydrogenation of chromanone is described below. A 50 mL stainless steel autoclave was charged with RuCl[(S,S)-Tsdpen](mesitylene) (1.0 mg, 0.0016 mmol) under argon. Then 4-chromanone (474 mg, 3.2 mmol) and methanol (6.4 mL) were added thereto. After pressurization with hydrogen, substitution was conducted five times. Hydrogen was charged to 50 atm to initiate reaction. After stirring for 23 hours at 30° C., the reaction pressure was reduced to normal. The product was analyzed by ¹H-NMR and HPLC reporting synthesis of (S)-4-chromanol in 91% ee and 100% yield.

### Example 46

An example of synthesizing optically active 4-chromanol by hydrogenation of chromanone is described below. A 50 mL stainless steel autoclave was charged with RuCl[(S,S)-Tsdpen](p-cymene) (1.0 mg, 0.0016 mmol) under argon. Then 4-chromanone (474 mg, 3.2 mmol) and methanol (6.4 mL) were added thereto. After pressurization with hydrogen, substitution was conducted five times. Hydrogen was charged to 50 atm to initiate reaction. After stirring for 23 hours at 30° C., the reaction pressure was reduced to normal. The product was analyzed by ¹H-NMR and HPLC reporting synthesis of (S)-4-chromanol in 97% ee and 85% yield.

### Example 47

An example of synthesizing optically active 4-chromanol by hydrogenation of chromanone is described below. A 50 mL stainless steel autoclave was charged with RuCl[(S,S)-Tsdpen](p-cymene) (1.0 mg, 0.0016 mmol) and NaClO₄ (10 mg, 0.08 mmol) under argon. Then 4-chromanone (1185 mg, 8.0 mmol) and methanol (16 mL) were added thereto. After pressurization with hydrogen, substitution was conducted five times. Hydrogen was charged to 50 atm to initiate reaction. After stirring for 23 hours at 30° C., the reaction pressure was reduced to normal. The product was analyzed by ¹H-NMR and HPLC reporting synthesis of (S)-4-chromanol in 97% ee and 93% yield.

### Example 48

An example of synthesizing optically active (3′-hydroxyphenyl)ethanol by hydrogenation of 3′-hydroxyacetophenone is described below. A 50 mL stainless steel autoclave was charged with RuCl[(S,S)-Tsdpen](mesitylene) (0.93 mg, 0.0015 mmol) and NaClO₄ (9.2 mg, 0.075 mmol) under argon. Then 3′-hydroxyacetophenone (613 mg, 4.5 mmol) and methanol (9 mL) were added thereto. After pressurization with hydrogen, substitution was conducted five times. Hydrogen was charged to 100 atm to initiate reaction. After stirring for 20 hours at 30° C., the reaction pressure was reduced to normal. The product was analyzed by ¹H-NMR and HPLC reporting synthesis of optically active (3′-hydroxyphenyl)ethanol in 98% ee and 98% yield.

### Example 49

An example of synthesizing optically active 5,6-dihydro-4H-thieno[2,3-b]thiopyran-4-hydroxy-7,7-dioxide by hydrogenation of 5,6-dihydro-4H-thieno[2,3-b]thiopyran-4-one-7,7-dioxide is described below. A 50 mL stainless steel autoclave was charged with RuCl[(S,S)-Tsdpen](mesitylene) (0.93 mg, 0.0015 mmol) and NaClO₄ (9.2 mg, 0.075 mmol) under argon. Then 5,6-dihydro-4H-thieno[2,3-b]thiopyran-4-one-7,7-dioxide (455 mg, 2.25 mmol) and methanol (22.5 mL) were added thereto. After pressurization with hydrogen, substitution was conducted five times. Hydrogen was charged to 100 atm to initiate reaction. After stirring for 24 hours at 30° C., the reaction pressure was reduced to normal. The product was analyzed by ¹H-NMR and HPLC reporting synthesis of (S)-5,6-dihydro-4H-thieno[2,3-b]thiopyran-4-hydroxy-7,7-dioxide in 98% ee and 100% yield.

### Example 50

An example of synthesizing optically active 1,2-propanediol by hydrogenation of acetol is described below. A 50 mL stainless steel autoclave was charged with RuCl[(S,S)-Tsdpen](mesitylene) (0.93 mg, 0.0015 mmol) and NaClO₄ (9.2 mg, 0.075 mmol) under argon. Then acetol (111 mg, 1.5 mmol) and methanol (3.0 mL) were added thereto. After pressurization with hydrogen, substitution was conducted five times. Hydrogen was charged to 100 atm to initiate reaction. After stirring for 17 hours at 30° C., the reaction pressure was reduced to normal. The product was analyzed by ¹H-NMR and HPLC reporting synthesis of (R)-1,2-propanediol in 63% ee and 97% yield.

### Example 51

An example of synthesizing optically active 2,3-butanediol by hydrogenation of 2,3-butanedione is described below. A 50 mL stainless steel autoclave was charged with RuCl[(S,S)-Tsdpen](p-cymene) (0.95 mg, 0.0015 mmol) and NaClO₄ (9.2 mg, 0.075 mmol) under argon. Then 2,3-butanedione (129 mg, 1.5 mmol) and methanol (3.0 mL) were added thereto. After pressurization with hydrogen, substitution was conducted five times. Hydrogen was charged to 50 atm to initiate reaction. After stirring for 18 hours at 30° C., the reaction pressure was reduced to normal. The product was analyzed by ¹H-NMR and HPLC reporting synthesis of (S,S)-2,3-butanediol in yield of 47%.

### Example 52

An example of synthesizing (R)-4-phenyl-3-butyn-2-ol by hydrogenation of 4-phenyl-3-butyn-2-one is described below. A 50 mL stainless steel autoclave was charged with a ruthenium complex, RuHCl[(S)-tolbinap][(S,S)-dpen] (1 mg, 0.00097 mmol). After argon substitution, 4-phenyl-3-butyn-2-one (0.283 mL, 1.94 mmol) and methanol (1.9 mL) were added thereto. Hydrogen was pressurized and substitution was conducted (five times). Hydrogen was charged to 9 atm to initiate reaction. After stirring for 11 hours at 30° C., the reaction pressure was reduced to normal, and the reaction solution was analyzed by ¹H-NMR and HPLC to determine the determinate quantity and optical purity of the product, i.e., 4-phenyl-3-butyn-2-ol. As a result, (R)-4-phenyl-3-butyn-2-ol was obtained in 74% ee and 65% yield. In the nomenclature of this ruthenium complex, the metal atom, the hydrogen atom, the anionic group, the diphosphine ligand, and the diamine ligand are presented in this order from the left (see formula (6) below).

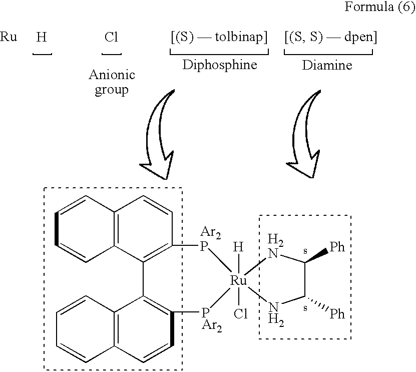

### Examples 53-54

Reaction was conducted as in EXAMPLE 52 but with a ruthenium complex, RuHCl[(S,S)-tolbinap][(S,S)-dpen] as a catalyst and with different reaction temperatures and additives. The results are shown in Table 6.

TABLE 6

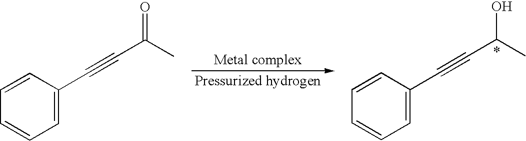

| Examples | chiral Ru cat | temp, ° C. | additive | yield (%) | ee (%) | config |
|---|---|---|---|---|---|---|
| 53 | RuHCl[(S)-tolbinap][(S,S)-dpen] | 50 | — | 100 | 75 | R |
| 54 | RuHCl[(S)-tolbinap][(S,S)-dpen] | 30 | NaClO₄ | 96 | 75 | R |
| | | | 0.05 mol | | | |
| 2^{a} | RuH(BH₄)[(S)-tolbinap][(S,S)-dpen] | 30 | NaClO₄ | 20 | 76 | R |
| | | | 0.05 mol | | | |

Conditions:
chiral Ru cat 0.001 mmol, CH₃OH 2 ml, S/C = 2000, time 11 h, H₂ 9 atm,
[ketone] = 1.0 M.
^{a}Comparative example

### Comparative Example 2

Reaction was conducted as in EXAMPLE 52 but with a ruthenium complex, RuH(BH4)[(S,S)-tolbinap][(S,S)-dpen], as a catalyst and with different reaction temperature and additive. The results are shown in Table 6.

### Comparative Example 3

A 50 mL stainless steel autoclave was charged with a ruthenium complex, RuCl₂[(S)-tolbinap][(S,S)-dpen] (1 mg, 0.00097 mmol) and KOt-Bu (0.1 mg, 0.00097 mmol). After argon substitution, 4-phenyl-3-butyn-2-one (0.283 mL, 1.94 mmol) and methanol (1.9 mL) were added thereto. Hydrogen was pressurized and substitution was conducted (five times). Hydrogen was charged to 9 atm to initiate reaction. After stirring for 11 hours at 30° C., the reaction pressure was reduced to normal, and the reaction solution was analyzed by ¹H-NMR, reporting generation of only trace amounts of 4-phenyl-3-butyn-2-ol.

### Comparative Example 4

Reaction of 4-phenyl-3-butyn-2-one was conducted as in COMPARATIVE EXAMPLE 2 but in 2-propanol. The reaction solution was analyzed by ¹H-NMR, reporting generation of only trace amounts of 4-phenyl-3-butyn-2-ol.

### INDUSTRIAL APPLICABILITY

The present invention is applicable to production of an optically active alcohol usable as an intermediate or the like of medicines, agricultural chemicals, and many general-purpose chemical agents.

1-10. (canceled)

11. A process for producing an optically active alcohol, comprising placing a metal complex represented by general formula (1) below and a ketone compound in a polar solvent and stirring the mixture under pressurized hydrogen to hydrogenate the ketone compound to thereby obtain the optically active alcohol: 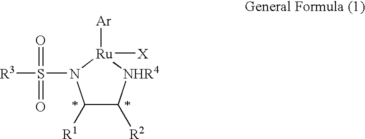
(where R¹ and R² may be the same or different and are each selected from the group consisting of an alkyl group, an optionally substituted phenyl group, an optionally substituted naphthyl group, and an optionally substituted cycloalkyl group, or together form an optionally substituted alicyclic ring;
R³ is one selected from the group consisting of an alkyl group, a perfluoroalkyl group, an optionally substituted naphthyl group, an optionally substituted phenyl group, and a camphor group;
R⁴ is a hydrogen atom or an alkyl group;
Ar is an optionally substituted benzene;
X is an anionic group; and
* represents an asymmetric carbon.)

12. A process for producing an optically active alcohol, comprising placing a metal complex represented by general formula (2) and a ketone compound in a polar solvent and stirring the mixture under pressurized hydrogen to hydrogenate the ketone compound to thereby obtain the optically active alcohol: 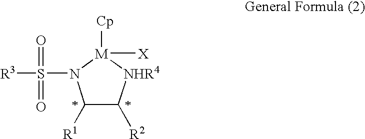
(where R¹ and R² may be the same or different and are each selected from the group consisting of an alkyl group, an optionally substituted phenyl group, an optionally substituted naphthyl group, and an optionally substituted cycloalkyl group, or together form an optionally substituted alicyclic ring;
R³ is one selected from the group consisting of an alkyl group, a perfluoroalkyl group, an optionally substituted naphthyl group, an optionally substituted phenyl group, and a camphor group;
R⁴ is a hydrogen atom or an alkyl group;
Cp is an optionally substituted cyclopentadiene;
M is rhodium or iridium;
X is an anionic group; and
* represents an asymmetric carbon.)

13. The process for producing the optically active alcohol according to claim 11, wherein in general formulae (1) and (2), R¹, R², and R³ may be the same or different and each represent a phenyl group, a phenyl group having a C₁-C₅ alkyl group, a phenyl group having a C₁-C₅ alkoxy group, or a phenyl group having a halogen substituent.

14. The process for producing the optically active alcohol according to claim 12, wherein in general formulae (1) and (2), R¹, R², and R³ may be the same or different and each represent a phenyl group, a phenyl group having a C₁-C₅ alkyl group, a phenyl group having a C₁-C₅ alkoxy group, or a phenyl group having a halogen substituent.

15. A process for producing an optically active alcohol, comprising placing a metal complex represented by general formula (3) and a ketone compound in a polar solvent and stirring the mixture under pressurized hydrogen to hydrogenate the ketone compound to thereby obtain the optically active alcohol: 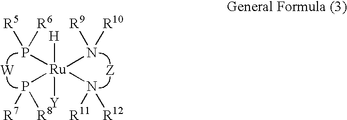
(where W is an optionally substituted bonding chain;
R⁵ to R⁸ may be the same or different and each represent an optionally substituted hydrocarbon group; R⁵ and R⁶ may bind each other to form an optionally substituted carbon chain ring; and R⁷ and R⁸ may bind each other to form an optionally substituted carbon chain ring;
R⁹ to R¹² may be the same or different and each represent a hydrogen atom or an optionally substituted hydrocarbon group;
Z is an optionally substituted hydrocarbon chain;
Y is an anionic group other than BH₄; and
each ligand of the ruthenium may be at any position.)

16. The process for producing the optically active alcohol according to claim 15, wherein, in general formula (3), W in R⁵R⁶P—W—PR⁷R⁸ is a binaphthyl group which is bonded to the phosphorus atoms at 2-position and 2′-position and which may have a substituent at any other position.

17. The process for producing the optically active alcohol according to claim 11, wherein the polar solvent is methanol or ethanol.

18. The process for producing the optically active alcohol according to claim 12, wherein the polar solvent is methanol or ethanol.

19. The process for producing the optically active alcohol according to claim 15, wherein the polar solvent is methanol or ethanol.

20. The process for producing the optically active alcohol according to claim 11, wherein no base is added.

21. The process for producing the optically active alcohol according to claim 12, wherein no base is added.

22. The process for producing the optically active alcohol according to claim 15, wherein no base is added.

23. The process for producing the optically active alcohol according to claim 11, wherein the ketone compound is unstable in the presence of bases.

24. The process for producing the optically active alcohol according to claim 12, wherein the ketone compound is unstable in the presence of bases.

25. The process for producing the optically active alcohol according to claim 15, wherein the ketone compound is unstable in the presence of bases.

26. The process for producing the optically active alcohol according to claim 11, wherein the ketone compound is a cyclic ketone, a ketone having an olefin moiety, a ketone having an acetylene moiety, a ketone having a hydroxyl group, a ketone having a halogen substituent, a chromanone derivative, a diketone, a ketoester, or a ketoamide.

27. The process for producing the optically active alcohol according to claim 12, wherein the ketone compound is a cyclic ketone, a ketone having an olefin moiety, a ketone having an acetylene moiety, a ketone having a hydroxyl group, a ketone having a halogen substituent, a chromanone derivative, a diketone, a ketoester, or a ketoamide.

28. The process for producing the optically active alcohol according to claim 15, wherein the ketone compound is a cyclic ketone, a ketone having an olefin moiety, a ketone having an acetylene moiety, a ketone having a hydroxyl group, a ketone having a halogen substituent, a chromanone derivative, a diketone, a ketoester, or a ketoamide.

29. The process for producing the optically active alcohol according to claim 11, wherein the ketone compound is a ketone compound having a halogen substituent at α-position or α,β-alkynyl ketone.

30. The process for producing the optically active alcohol according to claim 12, wherein the ketone compound is a ketone compound having a halogen substituent at α-position or α,β-alkynyl ketone.

31. The process for producing the optically active alcohol according to claim 15, wherein the ketone compound is a ketone compound having a halogen substituent at α-position or α,β-alkynyl ketone.
